(19) United States
(12) Patent Application Publication
(10) Pub. No.: US 2022/0280613 A1
(43) Pub. Date: Sep. 8, 2022

(54) COMPOSITIONS AND METHODS FOR TREATING ALZHEIMERS DISEASE

(71) Applicant: UNIVERSITY OF SOUTH FLORIDA, TAMPA, FL (US)

(72) Inventor: Chuanhai Cao, Tampa, FL (US)

(21) Appl. No.: 17/626,205

(22) PCT Filed: Jul. 10, 2020
(86) PCT No.: PCT/US20/41719

§ 371 Date: Jan. 11, 2022

Related U.S. Application Data
(60) Provisional application No. 62/873,566, filed on Jul. 12, 2019.

(51) Int. Cl. A61K 38/28; A61K 31/352; A61K 31/4045; A61P 25/28; A61K 9/107; A61K 47/24; A61K 47/10; A61K 47/14

(52) U.S. Cl. CPC A61K 38/28; A61K 31/352; A61K 31/4045; A61P 25/28; A61K 9/1075; A61K 47/24; A61K 47/10; A61K 47/14

(57) ABSTRACT

The present application relates to compositions comprising (i) THC; (ii) melatonin; and (iii) insulin and methods of using same to Alzheimer's disease in a subject in need thereof without the psychological impairment and side effects associated with THC.

### CROSS-REFERENCE TO RELATED APPLICATIONS

This application claims priority to U.S. Provisional Application No. 62/873,566, filed on Jul. 12, 2020, which is incorporated by reference herein in its entirety.

### TECHNICAL FIELD

The present application relates to compositions and methods for treating Alzheimer's disease.

### BACKGROUND

Alzheimer's disease (AD) is one of the most common neurodegenerative disorders worldwide. In 2011 alone, 15 million family members have provided more than 17.4 billion hours of care to diagnosed Alzheimer's disease (AD) patients. That care translates into more than $210 billion of AD-related services (Alzheimer's, Assn, 2012 Alzheimer's disease facts and figures. Alzheimer's Dement. 2012; 8: 131-168). This disease translates into an enormous burden on caregivers, as well as the health care system, both medically and economically. To date, there have been no effective treatments developed to cure or delay the progression of AD (Saxena, Bioenergetics breakdown in Alzheimer's disease: Targets for new therapies. Int J Physiol Pathophysiol Pharmacol. 2011; 3: 133-139; Götz, et al., Modes of Aβ toxicity in Alzheimer's disease. Cell Mol Life Sci. 2011; 68: 3359-3375). By 2050, an estimated 11 to 16 million Americans will be living with the disease (Alzheimer's, Assn, 2012 Alzheimer's disease facts and figures. Alzheimer's Dement. 2012; 8: 131-168; Brookmeyer, et al, Forecasting the global burden of Alzheimer's disease. Alzheimers Dement. 2007; 3: 186-191). Thus, there is a significant unmet need for an effective treatment for Alzheimer's disease.

### SUMMARY OF THE INVENTION

Some embodiments provide compositions comprising (i) tetrahydrocannabinol (THC); (ii) melatonin; and (iii) insulin; or a pharmaceutically acceptable salt of any of the foregoing; and at least one excipient.

Some embodiments provide methods of treating Alzheimer's disease comprising administering a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient.

Some embodiments provide methods of intranasally administering a composition comprising (i) tetrahydrocannabinol (THC); (ii) melatonin; and (iii) insulin; or a pharmaceutically acceptable salt of any of the foregoing; and at least one excipient; and wherein the administering comprises an intranasal delivery device.

### DESCRIPTION OF DRAWINGS

FIG. 1A is a table depicting attributes of a non-transgenic (NTG) mouse control (Ctr) group. FIG. 1B is a table depicting attributes of a transgenic (TG) mouse control (Ctr) group. FIG. 1C is a table depicting attributes of NTG and TG mouse groups that each received a formulation containing THC, melatonin, and insulin (TMI formulation).

FIG. 2A is a plot of trial 1 (T1) and trial 5 (T5) errors of NTG and TG mice in a radial arm water maze (RAWM) pre-behavior test. FIG. 2B is a plot of T1 and T5 escape latency of NTG and TG mice in a radial arm water maze (RAWM) pre-behavior test.

FIG. 3A is a bar graph depicting plasma amyloid beta concentrations of the TG Ctr group and the TG TMI group. FIG. 3B is a bar graph depicting the number of errors observed in the NTG Ctr group, the NTG TMI group, the TG Ctr group, and the TG TMI group in a RAWM pre-behavior test.

FIG. 4A is a series of plots of T1 and T5 errors of NTG Ctr, NTG TMI, TG Ctr, and TG TMI mice groups in a RAWM post-behavior test. FIG. 4B is a series of plots of T1 and T5 latency of NTG Ctr, NTG TMI, TG Ctr, and TG TMI mice groups in a RAWM post-behavior test.

FIG. 5A is a table showing amyloid beta 40 (Aβ40) and amyloid beta 42 (A4132) plasma concentrations and RAWM errors. FIG. 5B is a table showing Aβ40 and Aβ42 plasma concentrations and RAWM latency.

FIG. 6A is a table showing changes in immune cell population for post-1.5 month behavior test. FIG. 6B is a table showing changes in immune cell population for post-3 month behavior test.

FIG. 7A is a bar graph showing CD3+/CD11c+ immune cell populations for NTG Ctr, NTG TMI, TG Ctr, and TG TMI mouse groups for post-1.5 month behavior test. FIG. 7B is a bar graph showing CD3+/CD8+ immune cell populations in NTG Ctr, NTG TMI, TG Ctr, and TG TMI groups for post-1.5 month behavior test. FIG. 7C is a bar graph showing CD3+/CD11c+ immune cell populations for NTG Ctr, NTG TMI, TG Ctr, and TG TMI mouse groups for post-3 month behavior test. FIG. 7D is a bar graph showing CD3+/CD8a+ immune cell populations in NTG Ctr, NTG TMI, TG Ctr, and TG TMI groups for post-3 month behavior test.

FIG. 8A is a table showing the number and gender of mice in the NTG Ctr, NTG TMI, TG Ctr, and TG TMI groups used in cytotoxic T cell population measurements for post 1.5-month behavior test. FIG. 8B depicts attributes of the mice used in the NTG Ctr, NTG TMI, TG Ctr, and TG TMI groups used in cytotoxic T cell population measurements for post 1.5-month behavior test.

### DETAILED DESCRIPTION

Alzheimer's disease (AD) is characterized by age-associated progressive memory decline. Two hallmarks of AD are amyloid beta and tau, which are associated with the development and progression of the disease. No new therapy for AD has been approved since 2003, and even the approved therapies have limited effectiveness. The present disclosure provides compositions and methods for treating AD.

### Definitions

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a linker” includes reference to one or more such linkers, and reference to “the cell” includes reference to a plurality of such cells.

The term “about” when referring to a number or a numerical range means that the number or numerical range referred to is an approximation, for example, within experimental variability and/or statistical experimental error, and thus the number or numerical range may vary ±10% of the stated number or numerical range.

As used herein the term “subject” is understood to include an animal, especially a mammal, and more especially a human.

As used herein the abbreviation “IU” refers to “insulin units.”

As used herein, the term “therapeutically effective amount” refers to that amount of a compound, combination of compounds, or composition sufficient to result in the amelioration of Alzheimer's disease or other neurodegenerative disorder or one or more symptoms thereof, prevent advancement of Alzheimer's disease or other neurodegenerative disorder, or cause regression of Alzheimer's disease or other neurodegenerative disorder.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or advancement of Alzheimer's disease. For purposes of the present disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” in some aspects also means prolonging survival as compared to expected survival if not receiving treatment. Those in need of treatment include those already with the condition or disorder and in some aspects further include those prone to have the condition or disorder.

In the context of Alzheimer's, the term “treating” includes any or all of: improvements in one or more of mood, cognition, and memory; reduced Aβ aggregation in the brain; and ameliorating one or more symptoms associated with the disease.

As used herein “psychological impairments and side effects” shall mean undesirable effects observed in subjects receiving THC in dose amounts of about 3 mg/kg and greater, including but not limited to feeling euphoric (“high”), a decrease in mitochondrial function, a decrease in APP protein levels, anxiety, paranoia, hippocampal neuronal loss and similar effects.

As used herein “nanoemulsion” is a heterogenous mixture including an organic phase dispersed in an aqueous phase, or an aqueous phase dispersed in an organic phase, wherein each phase of the emulsion is in the form of discrete droplets each having a diameter of from about 5 to about 200 nm.

### Compositions

Some embodiments provide compositions comprising (i) tetrahydrocannabinol (THC); (ii) melatonin; and (iii) insulin; or a pharmaceutically acceptable salt of any of the foregoing; and at least one excipient.

In some embodiments, the composition comprises about 5 μg to about 20 mg THC. In other embodiments, the composition comprises about 25 μg to about 1 mg THC. In still other embodiments, the composition comprises about 50 μg to about 5 mg THC. In some embodiments, the composition comprises about 100 μg to about 10 mg THC. In other embodiments, the composition comprises about 500 μg to about 15 mg THC. For example, about 5 μg, 10 μg, 20 μg, 25 μg, 50 μg, 100 μg, 250 μg, 500 μg, 750 μg, 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, or any value in between.

In some embodiments, the composition comprises about 1 mg to about 150 mg melatonin. In other embodiments, the composition comprises about 1 mg to about 25 mg melatonin. In still other embodiments, the composition comprises about 10 mg to about 50 mg melatonin. In some embodiments, the composition comprises about 25 mg to about 100 mg melatonin. In other embodiments, the composition comprises about 50 mg to about 150 mg melatonin. For example, about 1 mg, 5 mg, 10 mg, 15 mg, 20 mg, 25 mg, 40 mg, 50 mg, 60 mg, 80 mg, 100 mg, 120 mg, 140 mg, 150 mg or any value in between.

In some embodiments, the composition comprises about 1 IU to about 50 IU of insulin. In other embodiments, the composition comprises about 1 IU to about 4 IU of insulin. In still other embodiments, the composition comprises about 2 IU to about 6 IU of insulin. In some embodiments, the composition comprises about 3 IU to about 8 IU of insulin. In other embodiments, the composition comprises about 4 IU to about 10 IU of insulin. For example, about 1 IU, 2 IU, 3 IU, 4 IU, 5 IU, 6 IU, 7 IU, 8 IU, 9 IU, 10 IU, 15 IU, 20 IU, 25 IU, 30 IU, 35 IU, 40 IU, 45 IU, or 50 IU, or any value in between.

In some embodiments, the composition comprises: (i) about 25 μg to about 1 mg THC; (ii) about 1 mg to about 150 mg melatonin; and (iii) about 1 IU to about 50 IU of insulin; and at least one excipient; wherein the composition is an emulsion formulated for intranasal administration. In some embodiments, the composition is an oil/water emulsion.

In some embodiments, the THC is organic THC, synthetic THC, Dronabinol, 49-THC, or THC-A. In some embodiments, the THC is Δ9-THC.

In some embodiments, the ratio of THC to melatonin is from about 1:1 to about 1:500. In some embodiments, the ratio of THC to melatonin is from about 1:1 to about 1:10. In some embodiments, the ratio of THC to melatonin is from about 1:1 to about 1:5. In some embodiments, the ratio of THC to melatonin is about 1:1, about 1:2, about 1:3, about 1:4, about 1:5, or any value in between. In some embodiments, the ratio of THC to melatonin is about 1:2.

In some embodiments, the ratio of THC to melatonin is from about 1:400 to about 1:4000. For example, about 1:400, 1:500, 1:600, 1:700, 1:800, 1:900, 1:1000, 1:1250, 1:1500, 1:1750, 1:2000, 1:2250, 1:2500, 1:2750, 1:3000, 1:3250, 1:3500, 1:3750, 1:4000, or any value in between.

In some embodiments, the ratio of THC to insulin is about 1:5 to about 1:50. For example, about 1:5, 1:6, 1:7, 1:8, 1:9, 1:10, 1:12, 1:13, 1:15, 1:20, 1:25, 1:30, 1:35, 1:40, 1:45, 1:50, or any value in between. For example, about 1:12 or 1:13.

The pharmaceutical compositions of the subject invention can be formulated according to known methods for preparing pharmaceutically useful compositions.

Furthermore, as used herein, the term “excipient” means any of the standard pharmaceutically acceptable carriers. The excipient can include diluents, adjuvants, preservatives, and vehicles, as well as implant carriers, and inert, non-toxic solid or liquid fillers, diluents, or encapsulating material that does not react with the active ingredients of the invention. Examples include, but are not limited to, phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Pharmaceutically acceptable excipients also include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Preservatives include, but are not limited to, antioxidants, antimicrobial agents, and chelating agents. For example, parabens (e.g., sodium methyl paraben and propylparaben sodium), sulphites, nitrites, benzoates, benzyl alcohol, chlorobutanol, phenol, cresols, sorbic acid, thiomersal, propylene glycol, and benalkonium chloride. Formulations are described in a number of sources that are well known and readily available to those skilled in the art. For example, Remington's Pharmaceutical Sciences (Martin E W [1995] Easton Pa., Mack Publishing Company, 19th ed.) describes formulations which can be used in connection with the subject invention.

The pharmaceutical compositions described herein can be administered orally, parenterally, pulmonarily, intraperitoneally, bucally or as an oral or nasal spray. The terms “parental” or “parenterally,” as used herein, refers to modes of administration which include intravenous, intramuscular, intraperitoneal, intrasternal, subcutaneous and intraarticular injection and infusion.

The composition can be formulated as a solid or as a liquid. Liquid dosage forms can include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof. In some embodiments, the liquid dosage form is administered intranasally. In some embodiments, the liquid dosage form is encased in a capsule for oral administration. The skilled artisan understands that when a liquid composition comprises oil and water components (such as an emulsion) or solid and liquid components (such as a suspension or dispersion), the certain additional formulation parameters, such as the inclusion of a buffer, or a particular pH value, refer to the water component (e.g., of an emulsion) and/or the liquid components (e.g., of a suspension or dispersion).

The compositions described herein may be formulated as emulsions (e.g., nanoemulsions), which may include one or more oils and/or organic solvents that form an organic phase in which the THC, melatonin, and insulin are dissolved or suspended, an aqueous phase, and one or more emulsifiers or surfactants. Further components may include, e.g., excipients (e.g., diluents, adjuvants, preservatives, and vehicles, as well as implant carriers, and inert, non-toxic solid or liquid fillers, diluents, or encapsulating material) as previously described. Oils and/or organic solvents include, but are not limited to, lecithin, medium chain triglycerides oil (MCT), hemp oil, and fish oil. Emulsifiers and/or surfactants include, but are not limited to, polyethylene glycol (e.g., PEG 400), glycerol, Pluronic F68, polysorbates (e.g., polysorbate 20), and ceteareth 20.

In some embodiments, the composition further comprises one or more solubilizing agents. Solubilizing agents can be present in the composition in an amount of about 5 wt. % to about 50 wt. %, about 10 wt. % to about 25 wt. %, or about 10 wt. % to about 20 wt. %. For example, the solubilizing agent can be present at about 5 wt. %, 10 wt. %, 15 wt. %, 20 wt. %, 25 wt. %, 30 wt. %, 35 wt. %, 40 wt. %, 45 wt. %., 50 wt. %, or any value in between.

In some embodiments, the composition further comprises one or more buffers. In some embodiments, the one or more buffers can include, but are not limited to, a citrate buffer, a lactate buffer, a phosphate buffer, a maleate buffer, a tartarate buffer, a succinate buffer, or an acetate buffer. In some embodiments, the buffer is one or more of lithium lactate, sodium lactate, potassium lactate, calcium lactate, lithium phosphate, sodium phosphate, potassium phosphate, calcium phosphate, lithium maleate, sodium maleate, potassium maleate, calcium maleate, lithium tartarate, sodium tartarate, potassium tartarate, calcium tartarate, lithium succinate, sodium succinate, potassium succinate, calcium succinate, lithium acetate, sodium acetate, potassium acetate, calcium acetate. lithium citrate monohydrate, sodium citrate monohydrate, potassium citrate monohydrate, calcium citrate monohydrate, lithium citrate dihydrate, sodium citrate dihydrate, potassium citrate dihydrate, calcium citrate dihydrate, lithium citrate trihydrate, sodium citrate trihydrate, potassium citrate trihydrate, calcium citrate trihydrate, lithium citrate tetrahydrate, sodium citrate tetrahydrate, potassium citrate tetrahydrate, calcium citrate tetrahydrate, lithium citrate pentahydrate, sodium citrate pentahydrate, potassium citrate pentahydrate, calcium citrate pentahydrate, lithium citrate hexahydrate, sodium citrate hexahydrate, potassium citrate hexahydrate, calcium citrate hexahydrate, lithium citrate heptahydrate, sodium citrate heptahydrate, potassium citrate heptahydrate, or calcium citrate heptahydrate. The buffer can be present in the composition in an amount of about 0.01 wt. % to about 5 wt. %.

In some embodiments, the formulation has a pH of about 2 to about 7. For example, the formulation can have a pH of about 3 to about 4. In some embodiments, the formulation has a pH of about 3.5.

In some embodiments, the composition further comprises one or more sweeteners. A sweetener can be added to the liquid formulation to make it less bitter or palatable, or both. Sweeteners suitable for inclusion in the formulation can include, both natural and artificial sweeteners. In some embodiments, the sweetener is an artificial sweetener and can include intense or high-intensity sweeteners. Intense sweeteners are commonly used as sugar substitutes or sugar alternatives as they are many times sweeter than sugar but contribute only a few to no calories when added to food. Exemplary intense sweeteners include sorbitol, sucrose, saccharins such as sodium saccharin, cyclamates such as sodium cyclamates, aspartame, sucralose, thaumatin, and acesulfam K. In some embodiments, the sweetener is a natural sugar. For example, sugars such as monosaccharides, disaccharides and polysaccharides can be used in the liquid formulations provided herein. The sugars can include xylose, ribose, glucose, mannose, galactose, fructose, dextrose, sucrose, maltose, partially hydrolyzed starch or corn syrup, and sugar alcohols such as sorbitol, xylitol, mannitol, glycerin, and combination thereof. In some embodiments, the liquid formulation further comprises a sweetener. The sweetener can include a sugar. For example, the sweetener can include sucrose.

In some embodiments, the sweetener can be present in the composition in an amount of about 5 wt. % to about 50 wt. %, about 10 wt. % to about 25 wt. %, or about 10 wt. % to about 20 wt. %. For example, the sweetener can be present at about 5 wt. %, 10 wt. %, 15 wt. %, 20 wt. %, 25 wt. %, 30 wt. %, 35 wt. %, 40 wt. %, 45 wt. %., 50 wt. %, or any value in between.

In some embodiments, the composition further comprises one or more flavoring agents. The one or more flavoring agents can include at least one of a natural flavoring agent, a natural fruit flavoring agent, an artificial flavoring agent, an artificial fruit flavoring agent, flavor enhancers, or mixtures thereof. Exemplary flavoring agents can be found, for example in US CFR 21 § 172.515 (Apr. 1, 2015), which is incorporated by reference in its entirety. For example, cinnamon, raspberry, orange, maple, butterscotch, glycyrrhiza (licorice) syrup, fruit, berry, vanilla, acacia syrup, coca, chocolate-mint, wild cherry, walnut, eriodictyon, bubblegum, grapefruit, lime, marshmellow, gurana, coffee, peach, lemon, fennel, apricot, honey, mint, wintergreen, and cherry. The flavoring agent can be present in the composition in an amount of about 0.01 wt. % to about 2 wt. %, about 0.01 wt. % to about 0.1 wt. %, or about 0.2 wt. % to about 0.5 wt. %. For example, the flavoring agent can be present in an amount of about 0.01 wt. %, 0.1 wt. %, 0.2 wt. %, 0.3 wt. %, 0.4 wt. %, 0.5 wt. %, 0.7 wt. %, 1.0 wt. %, 1.5 wt. %, or 2.0 wt. %. In some embodiments, the flavoring agent can be present in the liquid formulation in an amount of about 0.5 wt. %.

In some embodiments, the composition further comprises one or more coloring agents.

In some embodiments, the composition is formulated for intranasal administration.

In some embodiments, the composition is formulated for oral administration.

In some embodiments, the composition is a suspension or an emulsion. In other embodiments, the composition is an emulsion. In some embodiments, the composition is a nanoemulsion. In some embodiments, the composition is an oil/water emulsion.

In some embodiments, the droplet size is from about 1 nm to about 500 nm, about 5 nm to about 250 nm, about 10 nm to about 200 nm, about 15 nm to about 150 nm, about 20 nm to about 100 nm, or any value in between. In some embodiments, the droplet size is from about 1 nm to about 20 nm, about 5 nm to about 50 nm, about 25 nm to about 75 nm, about 50 nm to about 100 nm, about 75 nm to about 150 nm, about 100 nm to about 200 nm, or any value in between.

In some embodiments, the droplet size present in the composition is a distribution from about 1 nm to about 500 nm, about 5 nm to about 250 nm, about 10 nm to about 200 nm, about 15 nm to about 150 nm, about 20 nm to about 100 nm, or any value in between. In some embodiments, the droplet size present in the composition is a distribution from about 1 nm to about 20 nm, about 5 nm to about 50 nm, about 25 nm to about 75 nm, about 50 nm to about 100 nm, about 75 nm to about 150 nm, about 100 nm to about 200 nm, or any value in between.

### Uses and Methods of Treatment

Some embodiments provide methods of treating Alzheimer's disease comprising administering a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient, as described herein.

Some embodiments provide methods of preventing or reducing pathological Tau seeding and/or spreading in a brain of a subject in need thereof, the method comprising administering to the subject a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient, as described herein.

Some embodiments provide methods of preventing or reducing pathological amyloid beta aggregate seeding and/or spreading in the brain of a subject in need thereof, the method comprising administering to the subject a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient, as described herein.

In some embodiments, the reduction is about 10% to about 100% (prevention of further seeding and/or spreading), about 10% to about 50%, about 25% to about 75%, about 50% to about 100%, or any value in between.

Some embodiments provide methods of increasing the number of CD8+ T cells and/or dendritic cells in the brain of a subject in need thereof, the method comprising administering to the subject a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient, as described herein.

In some embodiments, the increase in the number of CD8+ T cells and/or dendritic cells in the brain of a subject is an increase of about 1.1-fold to about 100-fold, about 1.1-fold to about 50-fold, about 1.1-fold to about 25-fold, about 1.1 fold to about 10-fold, about 1.1 to about 5-fold, about 10-fold to about 40-fold, about 25-fold to about 60-fold, about 40-fold to about 75-fold, about 60-fold to about 90-fold, about 75-fold to about 100-fold, or any value in between.

In some embodiments, the subject in need thereof has been diagnosed with AD. In some embodiments, the subject in need thereof is at risk for developing AD. In some embodiments, the subject in need thereof is suspected of suffering from AD.

In some embodiments, the composition does not cause measureable psychological impairments and side effects associated with high doses of THC.

In some embodiments, the total THC administered to the subject in need thereof per dose is at least 0.2 μg/kg of body weight of the subject. In other embodiments, the total THC administered to the subject in need thereof per dose is from about 0.2 μg/kg to about 0.16 mg/kg of body weight of the subject. In still other embodiments, the total THC administered to the subject in need thereof per dose is from about 0.2 μg/kg to about 0.02 mg/kg of body weight of the subject. For example, about 0.2 μg/kg, 0.5 μg/kg, 1 μg/kg, 2 μg/kg, 5 μg/kg, 10 μg/kg, 20 μg/kg, 40 μg/kg, 60 μg/kg, 80 μg/kg, 100 μg/kg, 120 μg/kg, 140 μg/kg, 160 μg/kg, or any value in between.

In some embodiments, the total melatonin administered to the subject in need thereof per dose is from about 0.11 mg/kg to about 1.1 mg/kg body weight of the subject. For example, about 0.11 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.00 mg/kg, 1.05 mg/kg, 1.10 mg/kg, or any value in between.

In some embodiments, the total insulin administered to the subject in need thereof per dose is from about 1 IU to about 50 IU of insulin. In other embodiments, the composition comprises about 1 IU to about 4 IU of insulin. In still other embodiments, the composition comprises about 2 IU to about 6 IU of insulin. In some embodiments, the composition comprises about 3 IU to about 8 IU of insulin. In other embodiments, the composition comprises about 4 IU to about 10 IU of insulin. For example, about 1 IU, 2 IU, 3 IU, 4 IU, 5 IU, 6 IU, 7 IU, 8 IU, 9 IU, 10 IU, 15 IU, 20 IU, 25 IU, 30 IU, 35 IU, 40 IU, 45 IU, or 50 IU, or any value in between.

In some embodiments, the composition is administered in 6 hour intervals; in 12 hour intervals; or in 24 hour intervals. In some embodiments, the composition is administered in 6 hour intervals. In other embodiments, the composition is administered in 12 hour intervals. In still other embodiments, the composition is administered in 24 hour intervals.

In some embodiments, the administration comprises intranasal administration.

In some embodiments, the composition administered to the subject in need thereof comprises: (i) about 25 μg to about 1 mg THC; (ii) about 1 mg to about 150 mg melatonin; and (iii) about 1 IU to about 50 IU of insulin; and at least one excipient.

In some embodiments, the THC is organic THC, synthetic THC, Dronabinol, 49-THC, or THC-A. In some embodiments, the THC is Δ9-THC.

In some embodiments, amyloid beta aggregation is reduced or eliminated in the subject. In certain embodiments, the amyloid beta comprises amyloid beta 40. In certain embodiments, the amyloid beta comprises amyloid beta 42. In some embodiments, the amyloid beta aggregation is reduced by about 25% to about 100% (elimination of detectable aggregation), for example, by about 25% to about 45%, about 35% to about 55%, about 45% to about 65%, about 55% to about 75%, about 65% to about 85%, about 75% to about 95%, about 25% to about 75%, about 50% to about 100%, or any value in between.

Some embodiments provide methods of intranasally administering a composition comprising (i) tetrahydrocannabinol (THC); (ii) melatonin; and (iii) insulin; or a pharmaceutically acceptable salt of any of the foregoing; and at least one excipient; and wherein the administering comprises an intranasal delivery device.

In some embodiments, the intranasal delivery device is a vapor inhaler, a rhinyle catheter, a multi-dose dropper, a single-dose dropper, a unit-dose pipette, a squeeze bottle, a multi-dose metered spray pump, a single-dose metered spray pump, an atomizer, or a nebulizer.

In some embodiments, devices for intranasal administration, include one or more features present in any inhalation device described herein. In some embodiments, devices for intranasal administration are spray devices. Suitable commercially available nasal spray devices include Accuspray™ (Becton Dickinson). In certain embodiments, spray devices for intranasal use are devices for which the performance of the device is not dependent upon the pressure applied by the user. These devices are known as pressure threshold devices. Pressure threshold devices release liquid from the nozzle only when a threshold pressure is applied. These devices make it easier to achieve a spray with a regular droplet size. Pressure threshold devices suitable for use with the present invention are known in the art and are described for example in WO 91/13281, EP 311863, and EP 516636. Pressure threshold devices are commercially available from Pfeiffer GmbH and are also described in Bommer, R. Pharmaceutical Technology Europe, September 1999.

In some embodiments, the intranasal devices can administer the compositions described herein by means of bi-dose delivery. Bi-dose devices contain two sub-doses of a single dose, one sub-dose for administration to each nostril. Generally, the two sub-doses are present in a single chamber and the construction of the device allows for efficient delivery of a single sub-dose at a time. Alternatively, a monodose device may be used for administering the compositions according to the invention.

### EXAMPLES

Materials and Methods

THC, Melatonin and Insulin were purchased from Sigma Aldrich. Oils and other molecules were also ordered from Sigma.

Mice for this study were ordered from Jax Laboratory Inc. and bred at University of South Florida (USF) animal facility and began pre-behavior tests (also referred to herein as “pre”) at 12 months old. Mice were caged individually and maintained on a 12/12-hour light/dark cycle in a temperature-controlled room. Food and water were available as much or as often as mice desired. Mice were 10 months of age when treatment began.

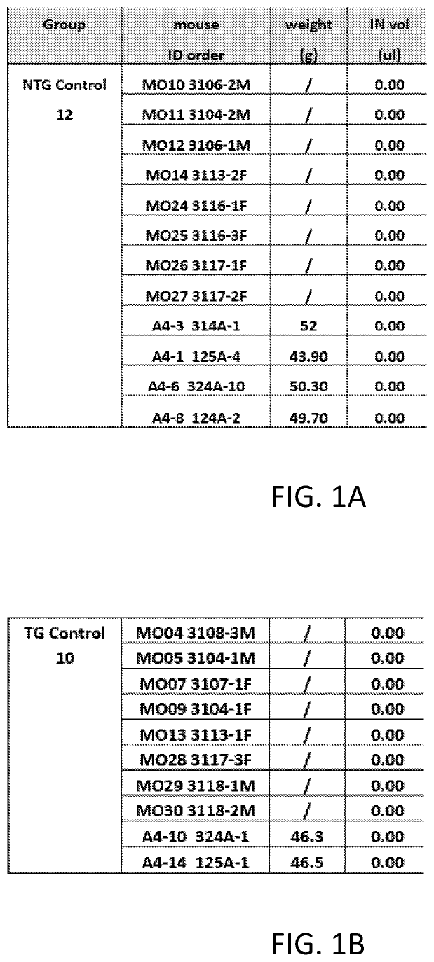

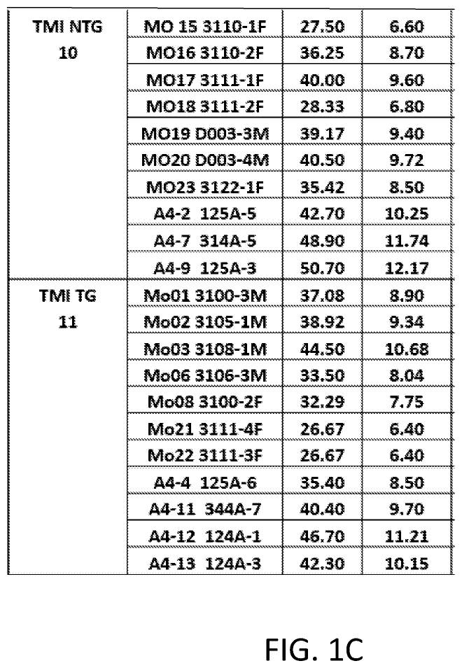

There were 43 mice at the beginning of this study, and the study ended at 27 mice (18 TG and 19 NTG). Weights and TMI formulation injection volumes of each mouse are listed in FIGS. 1A-1C. “Control” is also referred to herein as “Ctr”. The details for each assay of the mice are as listed: Plasma are collected (Pre: NTG Ctr n=11, NTG TMI n=9, TG Ctr n=10, TG TMI n=10; Post-1.5 months: NTG Ctr n=7, NTG TMI n=9, TG Ctr n=6, TG TMI n=10; Post-3 months: NTG Ctr n=11, NTG TMI n=9, TG Ctr n=10, TG TMI n=10); Brain soluble & insoluble Aβ (NTG Ctr n=11, NTG TMI n=9, TG Ctr n=10, TG TMI n=10. Flow cytometry Post-1.5 Months: NTG Ctr n=9, NTG TMI n=9, TG Ctr n=10, TG TMI n=10; Post-3 months: NTG Ctr n=10, NTG TMI n=9, TG Ctr n=10, TG TMI n=9); Behavior test for long term RAWM1-15 Days (Pre-behavior test: Combine cohort1-4 (NTG Ctr n=8, NTG TMI n=7, TG Ctr n=8, TG TMI n=7); Post-behavior test: Combine cohort1-5 (NTG Ctr n=9, NTG TMI n=9, TG Ctr n=10, TG TMI n=9).

Nanoemulsion preparation: To prepare the nano-emulsion formulation, the oil phase mixture was first prepared. 1.0 g of Lecithin (Fisher, 03376-250, Lot 153621) was weighed out. 2 mL medium chain triglycerides oil (provided by Now Sports, 100% pure, also referred to herein as “MCT”) was added to the Lecithin to yield the oil phase mixture. 2 mL of fish oil (Carlson the Finest Norwegian Fish oil, lemon taste) was added to the oil phase mixture. The oil phase mixture was then stirred at 200 rpm in a 500 mL glass beaker at 37° C. for 2 hours using a stir mixer until all added ingredients dissolved. The beaker was covered with plastic wrap. Once the ingredients of the oil phase mixture dissolved, the oil phase mixture was stirred for about 12 hours (e.g. overnight) at room temperature. Then, Δ9-THC, melatonin and insulin were added to the oil phase mixture and the oil phase mixture was then stirred at 200 rpm at room temperature for 1 hour. The water phase mixture was then prepared by combining in a 50 mL conical tube 1 mL polyethylene glycol (PEG 400) (Sigma P-3265, lot 81K0326), 0.5 mL glycerol (Sigma G5516-1L, lot SHBD3108V), 0.2 g sodium methyl paraben (Pfaltz and Bause, item number 506130, lot 18552, cas #5026-62-0), 0.2 g propylparaben sodium (Spectrum P1457, lot 2EC0375), 0.2 g Pluronic F68 (MP Biomedicals, A1288.0500, lot 5X010736, CAS 9003-11-6), and enough water to make the total volume of the water phase mixture reach 14 mL. The water phase mixture was shaken at room temperature for 30 minutes.

The oil phase mixture and the water phase mixture, both prepared as described above, were then combined in a 50 mL conical tube to yield an emulsion composition. Water was added to the resulting emulsion composition to bring the total volume of the emulsion composition to 20 ml. The emulsion composition was then vortexed (VWR Model: G560) for 20 minutes at maximum speed. The emulsion composition was then emulsified by three 11-minute cycles of sonication in an ice bath, allowing the emulsion composition to cool between each 11-minute cycle. The sonication cycles produced the nano-emulsion formulation. The sonication protocol was 54 s pulse on and 6 s pulse off, with an amplitude of 90%. The sonicator was Model FB120 from Fisher Scientific: 120 W, 120V, 50/60 Hz NOM, Frequency: 20 kHz, Full Size: 3 A SLO-BLD.

Drug delivery: Mice were restrained by hand, and 6 μL of the nanoemulsion was dropped into the nostrils at 2 μL three times at 1-minute intervals daily for 1.5 months or for three months.

Blood collection and processing: Blood was collected by mandibular vein punctuation with EDTA tube, and 10 μL were used for flow cytometry assay. The results of the blood were centrifuged at 1000 rcf for plasma collection, then plasma was frozen at −80° C. for future application.

Radial Arm Water Maze (RAWM): The radial arm water maze test was used to monitor the cognitive function of the mice before and after vaccinations. It contained six swim paths (arms) radiating from an open central area with a hidden escape platform located at the end of one of the arms. The pool was surrounded by several extra maze cues to for allow spatial navigation. On each trial, the mouse was allowed to swim for up to 60 seconds to find the escape platform. The platform was located in the same arm on each trial. On day one, mice were given 15 trials, alternating between a visible platform (above the water) and a hidden platform (below the water). On day two, mice were given 15 additional trials, with all the trials using a hidden platform. The starting arm varied for each trial so that the mice relied upon spatial cues to solve the task instead of learning motor rules. The goal arm for each mouse was different to avoid odor cues from revealing the goal arm. Entry into an incorrect arm (all four limbs within the arm) was scored as an error. Failure to make an arm entry within 15 seconds was also scored as an error. The errors for blocks of three consecutive trials were averaged for data analysis. Mice averaging one error or less by the end of day two are considered to have reached the learning criterion. On the third day, a reversal trial was performed, with the goal platform placed in the arm 180° from the original location. Mice were given 15 trials, all with a hidden platform. Latency to find and ascend the platform was recorded (60 s maximum).

Brain Tissue Preparation: After the RAWM test, mice were anesthetized with SomnaSol (Henry Schein Animal Health, Cat #024352) and intracardially perfused with 50 mL of saline. The brains were carefully removed, then the right hemisphere was frozen at −80° C. On the day of final brain tissue preparation, frozen tissue was thawed and homogenized in the RAPI buffer containing proteinase inhibitor (100 mM Tris [46], 150 mMNaCl, 0.5% DOC, 1% NP-40, 0.2% SDS, 1 mM Na3VO4, 10 mM NaF, 1 mM PMSF, 20 uM Leupeptin) with a pellet pestle motor and 10-seconds sonication, then centrifuged for 20 min at 21,000 g at 4° C. Crude protein concentrations were determined by Bio-rad DC protein assay (Bio-Rad Cat: 5000112) and adjusted to the same level for all the samples. The supernatants obtained from this protocol were stored at −80° C. The left hemispheres were transferred into a 4% paraformaldehyde solution for future immunohistochemistry tests.

Plasma and brain Aβ1-40/1-42 Level Detections: The concentrations of Aβ40/42 were measured by the Aβ1-40 and 1-42 specific sandwich ELISA kit (Mega Nano Biotech. FL, USA). In brief, each well of a 96 well plate was coated by 50 μl G1-42 (goat anti-human Aβ 1-42) antibody (Megananobiotech, FL, AB-001) diluted to 1×PBS 10 μg/mL and incubated overnight at 4° C. The plate was washed 5 times and blocked by adding 200 μl blocking buffer at 37° C. for 1 hour. After washing the plate, 50 μl diluted detection antibodies were mixed with either 50 μl diluted peptide standard (Aβ1-40 (Megananobiotech, FL, AB40-std) or 1-42 (Megananobiotech, FL AB42-std)) solution or diluted samples in a preparation plate and were then added into each well of the assay plate. Plates were then incubated at 4° C. overnight. After washing, 100 μl diluted secondary antibody was added to each well and incubated for 45 minutes on an orbital shaker at room temperature. The plate was washed 4 times, and TMB peroxidase substrate (Surmodics Cat: TMBS-1000) was added to each well and incubated at room temperature for 10 minutes. The reaction was stopped by adding 100 μl/well of 0.4M H2SO4. Absorbance at 450 nm was read with a BioTek Synergy H4 microplate reader. The concentration was calculated upon the peptide standard.

Statistical Analysis: The data were analyzed by using GraphPad Prism 6 software. Statistical evaluation of the results was initially performed using a one-way ANOVA involving all groups. This was then followed by a post-hoc pairwise analysis of group differences, and then Fisher LSD test was used. Pearson correlation was used to analyze the correlation. The level of statistical significance was set at α=0.05. Results were presented as mean±SEM.

### Example 1: Preparation of Nanoemulsion

The nanoemulsion was preparing according to the formulation in the table below and the following procedure.

| | Component | Quantity | | Volume |
|---|---|---|---|---|
| Oil | MCT oil | 1.875 | mL | 4.5 mL |
| Phase | Hemp | 1.875 | mL | |
| | Lecithin | 0.75 | g | |
| | Melatonin | 2.5 | mg | |
| | THC | 8.53 μL (1245 μg) | | |
| Water | Methyl Paraben | 0.15 | g | Add water to |
| Phase | (Pfaltz & Bause | | | 9.195 mL |
| | 5026-62-0) | | | |
| | Propyl Paraben | 0.15 | g | |
| | (Pfaltz & Bause | | | |
| | 35285-69-9) | | | |
| | Pluronic F 68 | 0.15 | g | |
| | (MP 2750016) | | | |
| | Glycerine | 0.375 | mL | |
| | (Fisher 56-81-5) | | | |
| | PEG 400 | 0.75 | mL | |
| | (Fisher p167-1) | | | |
| Insulin | Insulin (28.8 U/mg) | 15.58 | mg | Add water and |
| | | | | HCl to 1.305 ml |
| | 12N HCl | 6.45 | μL | |
| Total | | | | 15.0 mL |

Oil Phase: medium chain triglyercide oil and lecithin were mixed in 10 mL beaker at 300 rpm for 2 hr at 60° C. Melatonin and THC were added following complete dissolution of lecithin, with one additional hour of stirring at 300 rpm and 60° C.

Insulin: 15.58 mg (28.8 U/mg) insulin was suspended in 1 mL H₂O and 5 μL 12N HCl, and water was added until complete dissolution of insulin (at about 1.3 mL total volume).

Water Phase: The following composition was preparing in a 40 mL beaker and mixed for 2 hours at 300 rpm and room temperature.

1% methylparaben=0.15 mg

1% propylparaben=0.15 mg

1% Pluronic F=0.15 mg

2.5% Glycerine=0.375 mL

5% PEG 400=0.75 mL

Water to total volume of about 9.19 mL

Once the individual components of the nanoemulsion were prepared, 6.128 mL of the water phase was transferred into a 20 mL plastic bottle containing 3 mL of the oil phase and 0.872 mL of the insulin solution. To form a primary emulsion, the mixture was vortexted three times for 10 minutes each. The mixture was then placed on ice and sonicated for 10 minutes at an output wattage about about 18-21, with pulses of 55 seconds on and 5 seconds off at 95% ampl. Following sonication, the mixture was cooled for 10 minutes. The sonication and cooling steps were then repeated three times. The resulting mixture was diluted 1:800 into water and the particle size was measured using Malvern Nano ZS90.

### Mouse Protocols

Mice for this study were ordered from Jax Laboratory Inc. and bred at University of South Florida (USF) animal facility and began pre-behavior tests (also referred to herein as “pre”) at 12 months old. Mice were caged individually and maintained on a 12/12-hour light/dark cycle in a temperature-controlled room. Food and water were available as much or as often as mice desired. Mice were 10 months of age when treatment began.

There were 43 mice at the beginning of this study, and the study ended at 27 mice (18 TG and 19 NTG). Weights and TMI formulation injection volumes of each mouse are listed in FIGS. 1A-1C. “Control” is also referred to herein as “Ctr”. The details for each assay of the mice are as listed: Plasma are collected (Pre: NTG Ctr n=11, NTG TMI n=9, TG Ctr n=10, TG TMI n=10; Post-1.5 months: NTG Ctr n=7, NTG TMI n=9, TG Ctr n=6, TG TMI n=10; Post-3 months: NTG Ctr n=11, NTG TMI n=9, TG Ctr n=10, TG TMI n=10); Brain soluble & insoluble Aβ (NTG Ctr n=11, NTG TMI n=9, TG Ctr n=10, TG TMI n=10. Flow cytometry Post-1.5 Months: NTG Ctr n=9, NTG TMI n=9, TG Ctr n=10, TG TMI n=10; Post-3 months: NTG Ctr n=10, NTG TMI n=9, TG Ctr n=10, TG TMI n=9); Behavior test for long term RAWM1-15 Days (Pre-behavior test: Combine cohort1-4 (NTG Ctr n=8, NTG TMI n=7, TG Ctr n=8, TG TMI n=7); Post-behavior test: Combine cohort1-5 (NTG Ctr n=9, NTG TMI n=9, TG Ctr n=10, TG TMI n=9).

Blood collection and processing: Blood was collected by mandibular vein punctuation with EDTA tube, and 10 μl were used for flow cytometry assay. The results of the blood were centrifuged at 1000 rcf for plasma collection, then plasma was frozen at −80° C. for future application.

Radial Arm Water Maze (RAWM): The radial arm water maze test was used to monitor the cognitive function of the mice before and after vaccinations. It contained six swim paths (arms) radiating from an open central area with a hidden escape platform located at the end of one of the arms. The pool was surrounded by several extra maze cues to for allow spatial navigation. On each trial, the mouse was allowed to swim for up to 60 seconds to find the escape platform. The platform was located in the same arm on each trial. On day one, mice were given 15 trials, alternating between a visible platform (above the water) and a hidden platform (below the water). On day two, mice were given 15 additional trials, with all the trials using a hidden platform. The starting arm varied for each trial so that the mice relied upon spatial cues to solve the task instead of learning motor rules. The goal arm for each mouse was different to avoid odor cues from revealing the goal arm. Entry into an incorrect arm (all four limbs within the arm) was scored as an error. Failure to make an arm entry within 15 seconds was also scored as an error. The errors for blocks of three consecutive trials were averaged for data analysis. Mice averaging one error or less by the end of day two are considered to have reached the learning criterion. On the third day, a reversal trial was performed, with the goal platform placed in the arm 180° from the original location. Mice were given 15 trials, all with a hidden platform. Latency to find and ascend the platform was recorded (60 s maximum).

Brain Tissue Preparation: After the RAWM test, mice were anesthetized with SomnaSol (Henry Schein Animal Health, Cat #024352) and intracardially perfused with 50 ml of saline. The brains were carefully removed, then the right hemisphere was frozen at −80° C. On the day of final brain tissue preparation, frozen tissue was thawed and homogenized in the RAPI buffer containing proteinase inhibitor (100 mM Tris [46], 150 mMNaCl, 0.5% DOC, 1% NP-40, 0.2% SDS, 1 mM Na3VO4, 10 mM NaF, 1 mM PMSF, 20 uM Leupeptin) with a pellet pestle motor and 10-seconds sonication, then centrifuged for 20 min at 21,000 g at 4° C. Crude protein concentrations were determined by Bio-rad DC protein assay (Bio-Rad Cat: 5000112) and adjusted to the same level for all the samples. The supernatants obtained from this protocol were stored at −80° C. The left hemispheres were transferred into a 4% paraformaldehyde solution for future immunohistochemistry tests.

Plasma and brain Aβ1-40/1-42 Level Detections: The concentrations of Aβ40/42 were measured by the Aβ1-40 and 1-42 specific sandwich ELISA kit (Mega Nano Biotech. FL, USA). In brief, each well of a 96 well plate was coated by 50 μl G1-42 (goat anti-human Aβ1-42) antibody (Megananobiotech, FL, AB-001) diluted to 1×PBS 10 μg/mL and incubated overnight at 4° C. The plate was washed 5 times and blocked by adding 200 μl blocking buffer at 37° C. for 1 hour. After washing the plate, 50 μl diluted detection antibodies were mixed with either 50 μl diluted peptide standard (Aβ1-40 (Megananobiotech, FL, AB40-std) or 1-42 (Megananobiotech, FL AB42-std)) solution or diluted samples in a preparation plate and were then added into each well of the assay plate. Plates were then incubated at 4° C. overnight. After washing, 100 μl diluted secondary antibody was added to each well and incubated for 45 minutes on an orbital shaker at room temperature. The plate was washed 4 times, and TMB peroxidase substrate (Surmodics Cat: TMBS-1000) was added to each well and incubated at room temperature for 10 minutes. The reaction was stopped by adding 100 μl/well of 0.4M H2SO4. Absorbance at 450 nm was read with a BioTek Synergy H4 microplate reader. The concentration was calculated upon the peptide standard.

Statistical Analysis: The data were analyzed by using GraphPad Prism 6 software. Statistical evaluation of the results was initially performed using a one-way ANOVA involving all groups. This was then followed by a post-hoc pairwise analysis of group differences, and then Fisher LSD test was used. Pearson correlation was used to analyze the correlation. The level of statistical significance was set at α=0.05. Results were presented as mean±SEM.

### Example 2: Tg Mice are Impaired in Memory Test Before Treatment

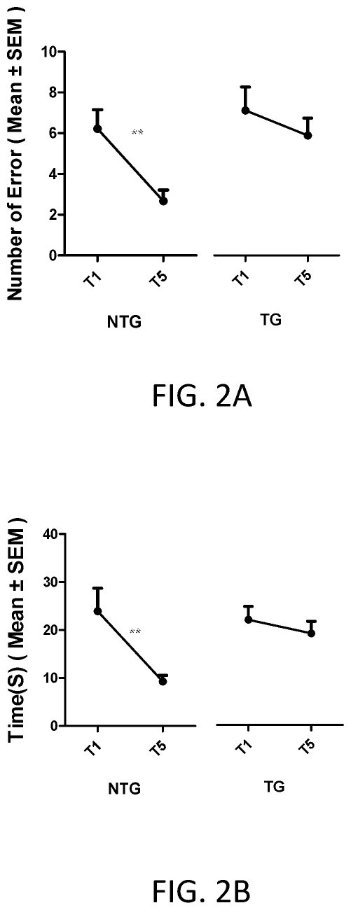

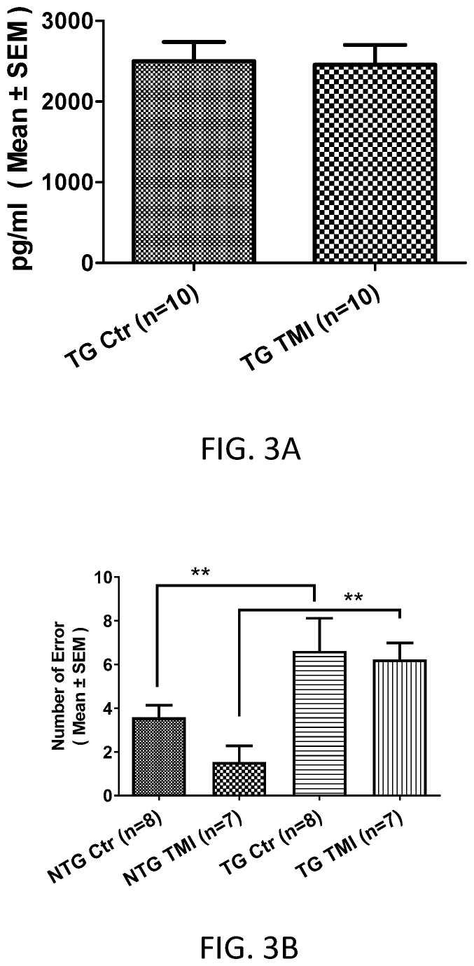

The memory of mice was tested using the RAWM, whose results demonstrated that the TG mice have memory impairment. The mice were grouped upon pre-treatment behavior results and blood amyloid beta level. FIGS. 2A-2B are the pretreatment behavior results for NTG and TG mice. FIG. 2A are plots depicting the errors the mice made in the RAWM swimming pool, and FIG. 2B are plots depicting the latency results of the mice finding the platform in the swimming pool of the RAWM. T1 and T5 indicate trial 1 and trial 5, respectively. NTG mice were significantly improved in trial 5 compared to trial 1, but there was no difference in TG mice. Thus, FIGS. 2A-2B demonstrated that TG mice have memory impairment. FIGS. 3A-3B depicts the grouping results based on the plasma Aβ level and pre-treatment behavior results. FIG. 3A shows that there were no differences between the TG Ctr and TG TMI groups. FIG. 3B is the pre-behavior grouping result, showing that there were significant differences between NTG Ctr and TG Ctr; there were also significant differences between the NTG TMI and TG TMI groups, but there were no differences between NTG Ctr and NTG TMI, as well as the TG Ctr and TG TMI groups. FIGS. 3A-3B show that mice were properly grouped into each study group.

### Example 3: TMI Treatment can Slow Down Memory Decline in TG Mice

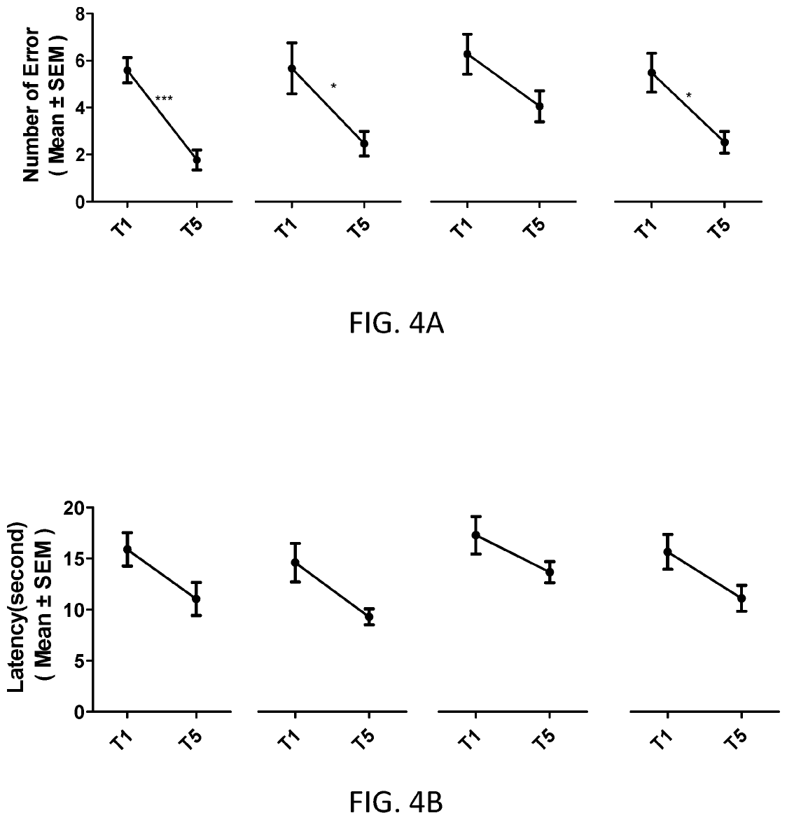

Memory tests were conducted after treatment with TMI for 3 months and demonstrated that TMI can reduce errors in both TG and NTG mice. FIGS. 4A-4B show behavior test results of post-treatment by RAWM. FIG. 4A depicts plots of the error test results for T1 vs. T5. In FIG. 4A, there is a significant difference between trial 1 versus trial 5 in the NTG Ctr (first plot), NTG TMI (second plot), and TG TMI (fourth plot) groups, but there are no differences in the TG Ctr (third plot) group. FIG. 4B is a series of plots depicting latency differences for the groups. In FIG. 4B, the first plot is of NTG Ctr, the second plot is of NTG TMI, the third plot is of TG Ctr, and the fourth plot is of TG TMI. FIG. 4B shows that there was no change in latency in all groups when comparing trial 1 to trial 5. (NTG Ctr n=9, NTG TMI n=9, TG Ctr n=10, TG TMI n=9).

These results demonstrate significant memory improvement after three months of daily treatment. The plasma amyloid and brain amyloid levels correlate highly to the performance of memory-related tasks.

### Example 4: Blood and Brain Amyloid Load are Correlated to Behavior Results

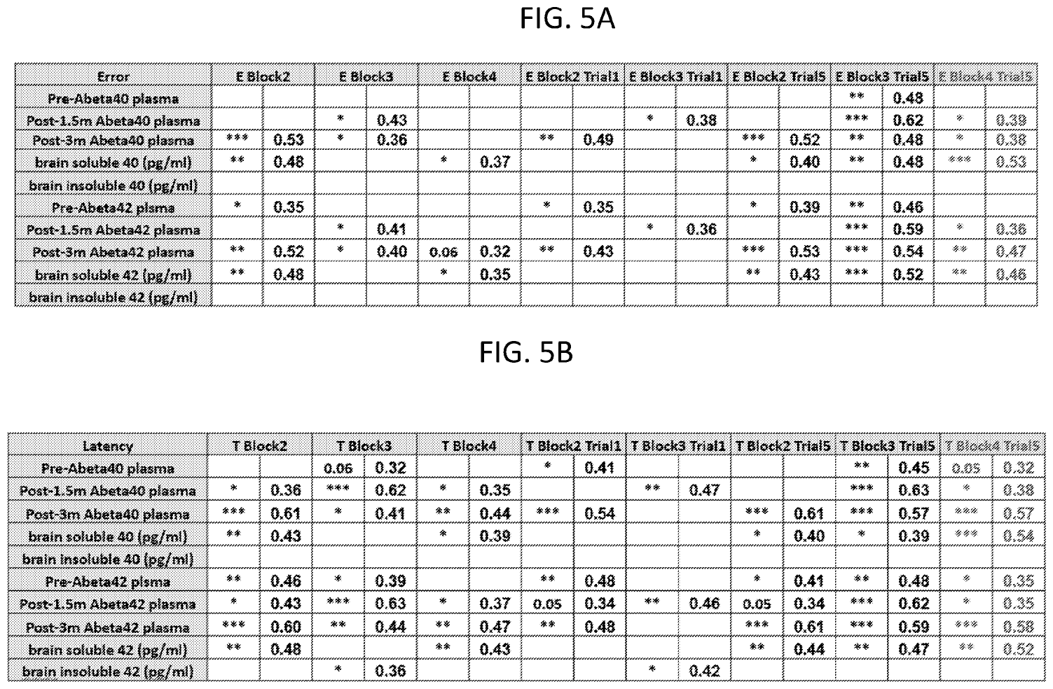

FIGS. 5A-5B depict tables showing correlation analysis results among Aβ level to behavior test for several blocks and trials. FIG. 5A depicts number of errors and FIG. 5B depicts latency. The correlation table demonstrated, e.g., that Aβ levels are higher for mice that have higher errors and latency in Block 4 Trial 5.

After three months of treatment on the APP/PS1 mouse model, it was observed that the TMI nanoemulsion can slow down memory impairment tested by RAWM. It was also shown that blood and brain Aβ level are correlated to behavior performance, with higher Aβ level correlated to higher errors and latency.

### Example 5: Immunological Effects

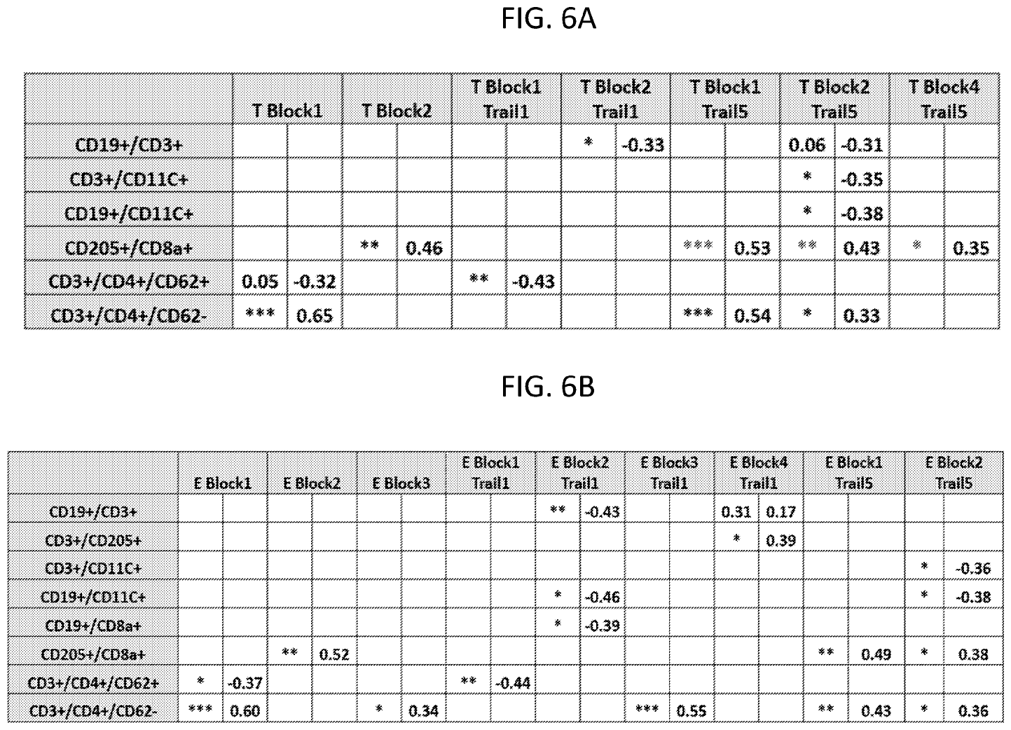

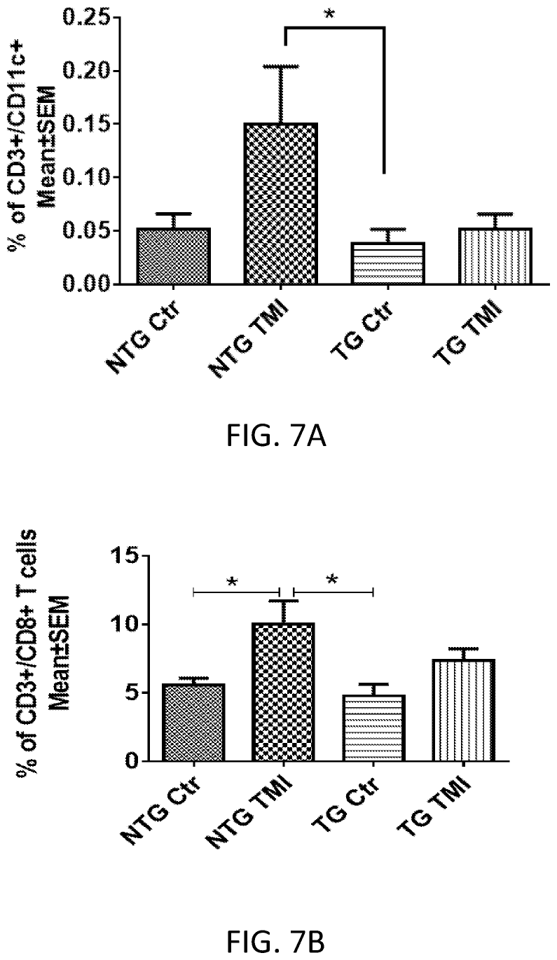

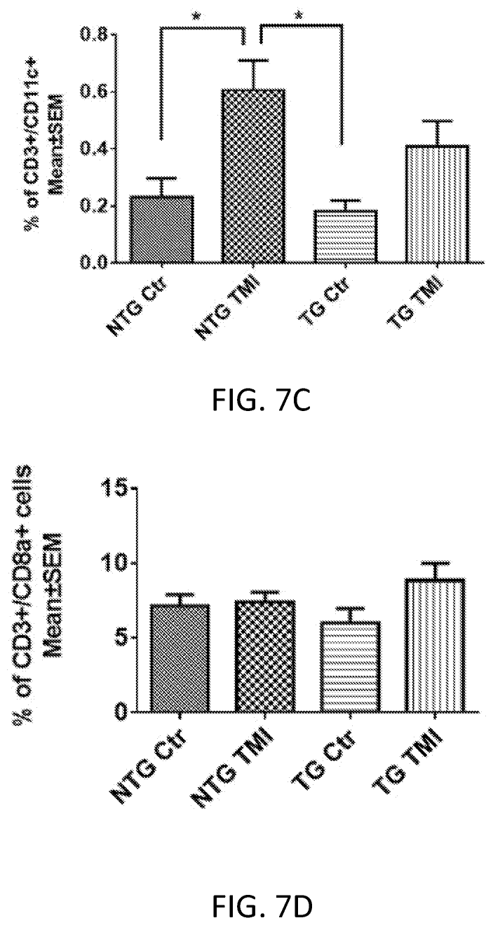

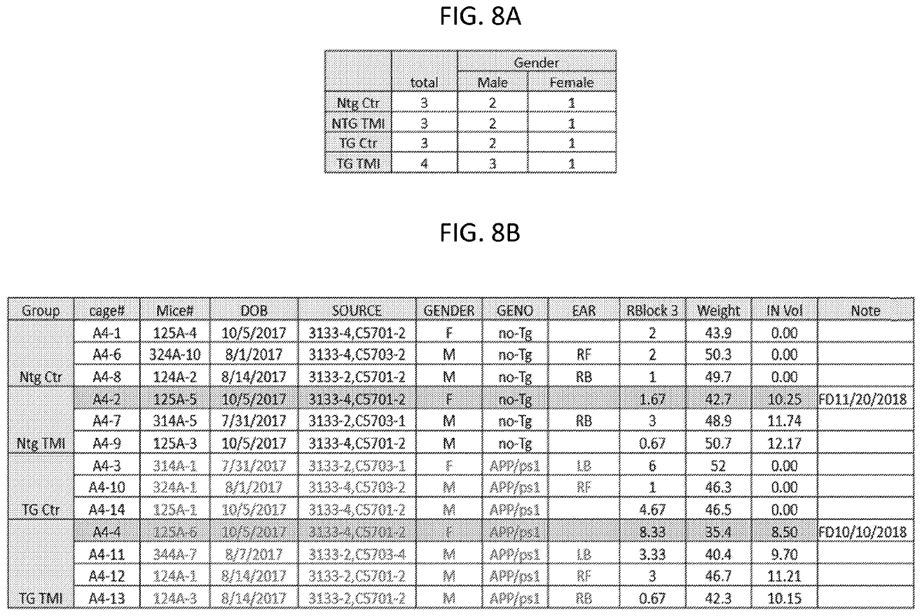

Immune cells in the peripheral blood were monitored and it was observed that the TMI formulation can modulate the immune system. FIG. 6A is a table showing changes in immune cell population for post-1.5 month behavior test. FIG. 6B is a table showing changes in immune cell population for post-3 month behavior test. FIG. 8A is a table showing the number and gender of mice in the NTG Ctr, NTG TMI, TG Ctr, and TG TMI groups used in cytotoxic T cell population measurements for post 1.5-month behavior test. FIG. 8B depicts attributes of the mice used in the NTG Ctr, NTG TMI, TG Ctr, and TG TMI groups used in cytotoxic T cell population measurements for post 1.5-month behavior test. FIGS. 7A-7D depict bar graphs showing the immunomodulatory effects of the nanoemulsion after delivery intranasally. FIGS. 7A-7B are bar graphs of results of flow cytometry for dendritic cells (FIG. 7A) and CD8+ T cells (FIG. 7B) after 1.5 months of treatment. There is a significant increase in both dendritic cells and CD8+ T cells in the NTG TMI groups compared to the NTG Ctr and TG Ctr groups, but no difference when the TG Ctr is compared to the TG TMI group after 1.5 months of treatment. FIGS. 7C-7D are bar graphs of results of flow cytometry for dendritic cells (FIG. 7C) and CD8+ T cells (FIG. 7D) after 3 months of treatment. There is a significant increase in the NTG TMI treatment group compared to NTG Ctr and TG Ctr group, but no differences between the NTG TMI and TG TMI groups. However, there are no differences in the CD8+ T cell populations among all groups after 3 months of treatment. These results demonstrate that the TMI has immunomodulatory effects through increasing dendritic cell populations and CD8 T cell population.

The disclosures of all publications cited herein are expressly incorporated herein by reference, each in its entirety, to the same extent as if each were incorporated by reference individually.

While there has been described and illustrated specific embodiments of a method of treating Alzheimer's disease, it will be apparent to those skilled in the art that variations and modifications are possible without deviating from the broad spirit and principle of the present invention. It is also to be understood that the following claims are intended to cover all of the generic and specific features of the invention herein described, and all statements of the scope of the invention which, as a matter of language, might be said to fall there between.

What is claimed is:

1. A composition comprising (i) tetrahydrocannabinol (THC); (ii) melatonin; and (iii) insulin; or a pharmaceutically acceptable salt of any of the foregoing; and at least one excipient.

2. The composition of claim 1, wherein the composition is formulated for intranasal administration.

3. The composition of claim 1 or 2, wherein the composition is a suspension or an emulsion.

4. The composition of any one of claims 1-3, wherein the composition is an emulsion.

5. The composition of any one of claim 1-4, wherein the composition is a nanoemulsion.

6. The composition of any one of claims 1-5, wherein the composition comprises about 5 μg to about 20 mg THC.

7. The composition of any one of claims 1-6, wherein the composition comprises about 25 μg to about 1 mg THC.

8. The composition of any one of claims 1-6, wherein the composition comprises about 50 μg to about 5 mg THC.

9. The composition of any one of claims 1-6, wherein the composition comprises about 100 μg to about 10 mg THC.

10. The composition of any one of claims 1-6, wherein the composition comprises about 500 μg to about 15 mg THC.

11. The composition of any one of claims 1-10, wherein the composition comprises about 1 mg to about 150 mg melatonin.

12. The composition of any one of claims 1-11, wherein the composition comprises about 1 mg to about 25 mg melatonin.

13. The composition of any one of claims 1-11, wherein the composition comprises about 10 mg to about 50 mg melatonin.

14. The composition of any one of claims 1-11, wherein the composition comprises about 25 mg to about 100 mg melatonin.

15. The composition of any one of claims 1-11, wherein the composition comprises about 50 mg to about 150 mg melatonin.

16. The composition of any one of claims 1-15, wherein the composition comprises about 1 IU to about 50 IU of insulin.

17. The composition of any one of claims 1-16, wherein the composition comprises about 1 IU to about 4 IU of insulin.

18. The composition of any one of claims 1-16, wherein the composition comprises about 2 IU to about 6 IU of insulin.

19. The composition of any one of claims 1-16, wherein the composition comprises about 3 IU to about 8 IU of insulin.

20. The composition of any one of claims 1-16, wherein the composition comprises about 4 IU to about 10 IU of insulin.

21. The composition of claim 1, comprising: (i) about 25 μg to about 1 mg THC; (ii) about 1 mg to about 150 mg melatonin; and (iii) about 1 IU to about 50 IU of insulin; and at least one excipient; wherein the composition is an emulsion formulated for intranasal administration.

22. The composition of any one of claims 1-21, wherein the THC is organic THC, synthetic THC, Dronabinol, Δ9-THC, or THC-A.

23. The composition of claim 22, wherein the THC is Δ9-THC.

24. The composition of any one of claims 1-23, wherein the ratio of THC to melatonin is from about 1:400 to about 1:4000.

25. A method of treating Alzheimer's disease comprising administering a composition to a subject in need thereof, wherein the composition comprises: (i) tetrahydrocannabinol (THC); (ii) melatonin; and (iii) insulin; or a pharmaceutically acceptable salt of any of the foregoing; and at least one excipient.

26. The method of claim 25, wherein the composition does not cause psychological impairments and side effects associated with high doses of THC.

27. The method of claim 25 or 26, wherein the total THC administered to the subject in need thereof per dose is at least 0.2 μg/kg of body weight of the subject.

28. The method of any one of claim 25-27, wherein the total THC administered to the subject in need thereof per dose is from about 0.2 μg/kg to about 0.16 mg/kg of body weight of the subject.

29. The method of any one of claim 25-28, wherein the total THC administered to the subject in need thereof per dose is from about 0.2 μg/kg to about 0.02 mg/kg of body weight of the subject.

30. The method of any one of claim 25-29, wherein the total melatonin administered to the subject in need thereof per dose is from about 0.11 mg/kg to about 1.1 mg/kg body weight of the subject.

31. The method of any one of claim 25-30, wherein the composition comprises about 1 IU to about 50 IU of insulin.

32. The method of any one of claim 25-31, wherein the composition comprises about 1 IU to about 4 IU of insulin.

33. The method of any one of claim 25-31, wherein the composition comprises about 2 IU to about 6 IU of insulin.

34. The method of any one of claim 25-31, wherein the composition comprises about 3 IU to about 8 IU of insulin.

35. The method of any one of claim 25-31, wherein the composition comprises about 4 IU to about 10 IU of insulin.

36. The method of any one of claim 25-35, wherein the composition is administered in 6 hour intervals; in 12 hour intervals; or in 24 hour intervals.

37. The method of any one of claim 25-36, wherein the administration comprises intranasal administration.

38. The method of claim 25, wherein the composition comprises: (i) about 25 ng to about 1 mg THC; (ii) about 1 mg to about 150 mg melatonin; and (iii) about 1 IU to about 50 IU of insulin; and at least one excipient.

36. The method of any one of claim 25-38, wherein the THC is organic THC, synthetic THC, Dronabinol, Δ9-THC, or THC-A.

36. The method of claim 36, wherein the THC is Δ9-THC.

37. The method of any one of claims 25-36, wherein amyloid beta is reduced or eliminated in the subject.

38. The method of claim 37, wherein the amyloid beta comprises amyloid beta 40.

39. The method of claim 38, wherein the amyloid beta comprises amyloid beta 42.
